(19) United States
(12) Patent Application Publication
(10) Pub. No.: US 2008/0075695 A1
(43) Pub. Date: Mar. 27, 2008

(54) COMBINATION THERAPY METHOD FOR TREATING HEPATITIS C VIRUS INFECTION AND PHARMACEUTICAL COMPOSITIONS FOR USE THEREIN

(76) Inventors: Anita Y.M. Howe, Paoli, PA (US); Stephen A. Villano, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/843,941

(22) Filed: Aug. 23, 2007

Related U.S. Application Data
(60) Provisional application No. 60/840,085, filed on Aug. 25, 2006.

(51) Int. Cl. A61K 38/21; A61P 31/12

(52) U.S. Cl. 424/85.5; 424/85.4; 424/85.6; 424/85.7

(57) ABSTRACT

Combination therapy methods for the treatment of hepatitis C virus infection and associated diseases, by the co-administration of 5-cyclopropyl-2-(4-fluoro-phenyl)-6-[(2-hydroxy-ethyl)-methanesulfonyl-amino]-benzofuran-3-carboxylic acid methylamide or a pharmaceutically acceptable salt thereof with natural, recombinant or modified interferon, that effectively inhibit viral replication.

### CROSS-REFERENCE TO RELATED APPLICATIONS

This application claims priority from U.S. Provisional Application No. 60/840,085, filed Aug. 25, 2006. The disclosure of the aforesaid application is incorporated by reference in its entirety in the present application.

### FIELD OF THE INVENTION

The present invention relates to a novel method for the treatment of hepatitis C viral infections by a therapeutic protocol wherein a specific benzofuran derivative and interferon are used in combination to inhibit viral replication.

### BACKGROUND OF THE INVENTION

Hepatitis C is a common infection that can lead to chronic hepatitis, cirrhosis, liver failure, and hepatocellular carcinoma. Infection with the hepatitis C virus (HCV) leads to chronic hepatitis in at least 85% of cases. It is the leading reason for liver transplantation, and is responsible for at least 10,000 deaths annually in the United States (Hepatology, 1997, 26 (Suppl. 1), 2S-10S).

Interferon (INF) and interferon in combination with ribavirin are used in the U.S. for hepatitis due to HCV. These treatments are associated with reduction in HCV-RNA levels and serum enzyme response in some patients. The remaining patients are non-responsive to treatment. For responders, a sustained clinical improvement is seen in only a small percentage of patients; the majority of patients relapse upon cessation of treatment. Thus, the effectiveness of therapy for chronic hepatitis C is variable and its cure rate remains low. Moreover, therapy is often associated with considerable side effects.

New therapies and preventatives are clearly needed for infections and diseases caused by the hepatitis C virus.

The hepatitis C virus is a member of the Flaviviridae family. The genome of HCV is positive strand, single stranded linear RNA (Hepatology, 1997, 26 (Suppl. 1), 11S-14S). HCV displays extensive genetic heterogeneity; at least six genotypes and more than 50 subtypes have been identified.

Following infection by HCV, the viral RNA is translated into a polyprotein. This approximately 3,000 residue polyprotein is subsequently cleaved into individual proteins by host peptidases, as well as virally encoded proteases. The HCV genome encodes structural proteins (required for virus assembly) and nonstructural proteins (required for replication). Some of the nonstructural proteins include: NS2, NS3, NS4A, NS4B, NS5A, and NS5B (J. General Virology, 2000, 81, 1631-1648). NS5B is a RNA-dependent RNA polymerase that is essential for viral replication. In positive stranded RNA viruses, such as HCV, RNA is the sole genetic material. Since mammalian host cells ordinarily lack RNA-dependent RNA polymerase activity, the positive stranded RNA viruses encode their own replicative polymerase (NS5B in the case of HCV), which is essential for the production of virion progeny. The inhibition of NS5B activity, therefore, provides an attractive target for HCV drug design.

Benzofuran compounds (BZFs), compositions and methods for treatment and prophylaxis of hepatitis C viral infections and associated diseases are disclosed in International Patent Application No. PCT/US2003/034962, published May 21, 2004 (WO 2004/041201), the entire disclosure of which is incorporated by reference herein.

### SUMMARY OF THE INVENTION

In accordance with one aspect, the present invention provides a method for treating HCV infection and diseases associated therewith in a patient in need of such treatment by administering a therapeutically effective combination of 5-cyclopropyl-2-(4-fluoro-phenyl)-6-[(2-hydroxy-ethyl)-methanesulfonyl-amino]-benzofuran-3-carboxylic acid methylamide and interferon. The combination therapy method includes the coordinated administration of the therapeutic agents in separate dosages, as well as administration of a pharmaceutical composition comprising a combined dosage.

Another aspect of the invention is a pharmaceutical composition comprising the above-mentioned benzofuran derivative and interferon in amounts effective for treating HCV infections, and diseases associated with such infections.

Still another aspect of the invention is an article of manufacture for coadministration of the therapeutic agents comprising two containers, one containing the above-mentioned benzofuran derivative, or a pharmaceutically acceptable salt with a pharmaceutically acceptable carrier, and another containing natural, recombinant or modified interferon with a pharmaceutically acceptable carrier.

### BRIEF DESCRIPTION OF THE DRAWINGS

FIG. 1. Plot of combination therapy clinical data showing mean change from baseline for all genotypes.

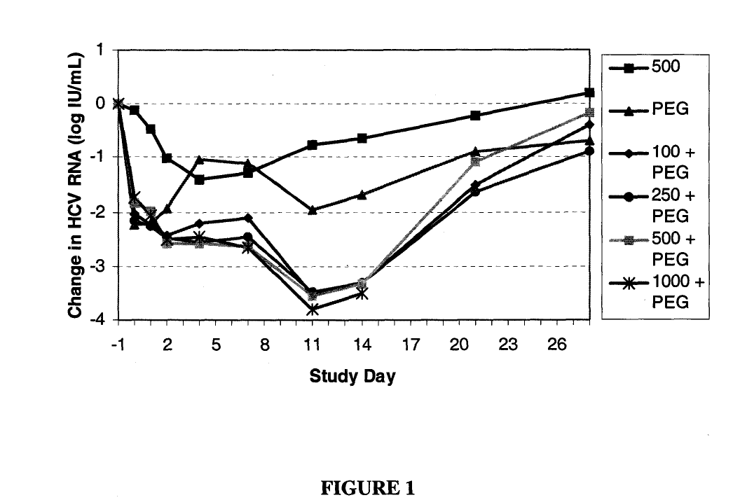

FIG. 2. Plot of combination therapy clinical data showing Mean Change, subtracting PEG-INF effect, for all genotypes.

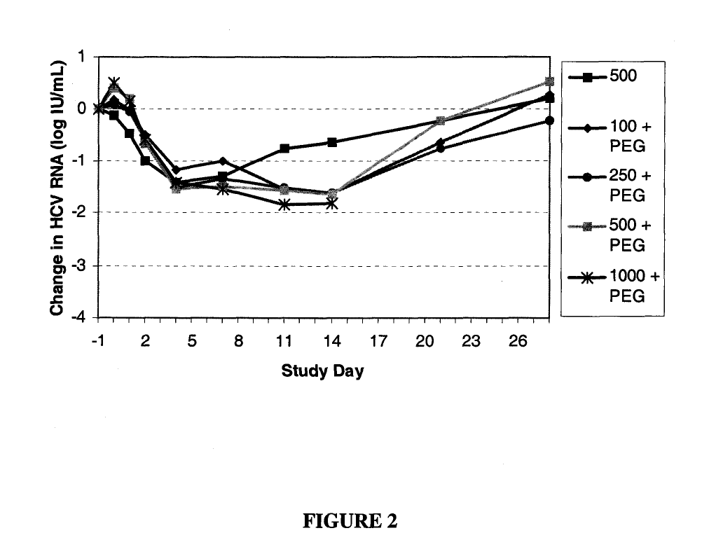

FIG. 3. Plot of combination therapy clinical data showing mean change, subtracting 500 mg BZF effect, for all genotypes.

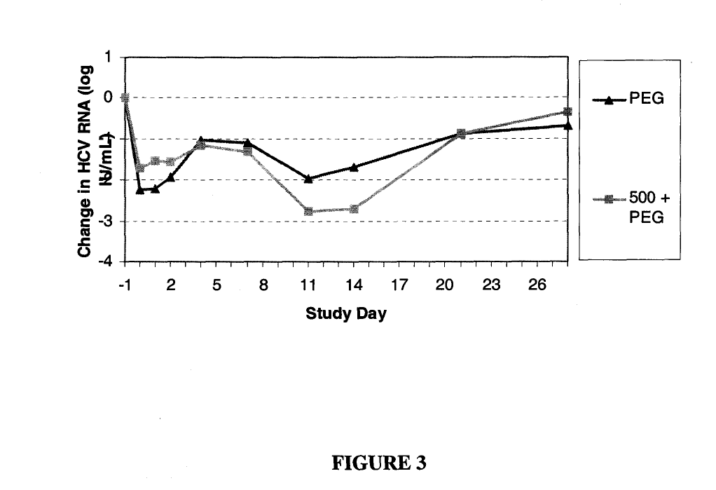

FIG. 4. Plot of combination therapy clinical data for 100 mg BZF dose showing mean change, compared to calculated additive effect for all genotypes.

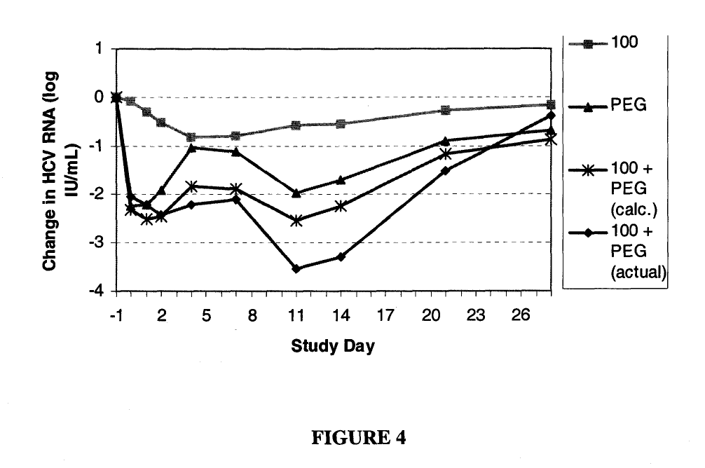

FIG. 5. Plot of combination therapy clinical data for 250 mg BZF dose showing mean change, compared to calculated additive effect for all genotypes

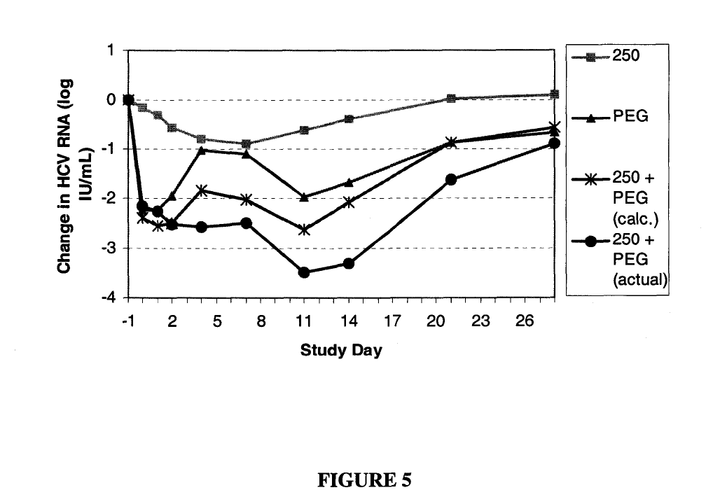

FIG. 6. Plot of combination therapy clinical data for 500 mg BZF dose showing mean change, compared to calculated additive effect for all genotypes

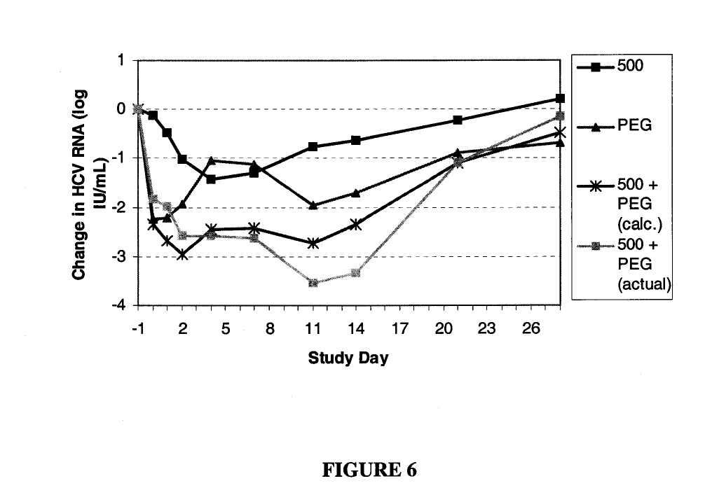

FIG. 7. Plot of combination therapy clinical data for 1000 mg BZF dose showing mean change, compared to calculated additive effect for all genotypes

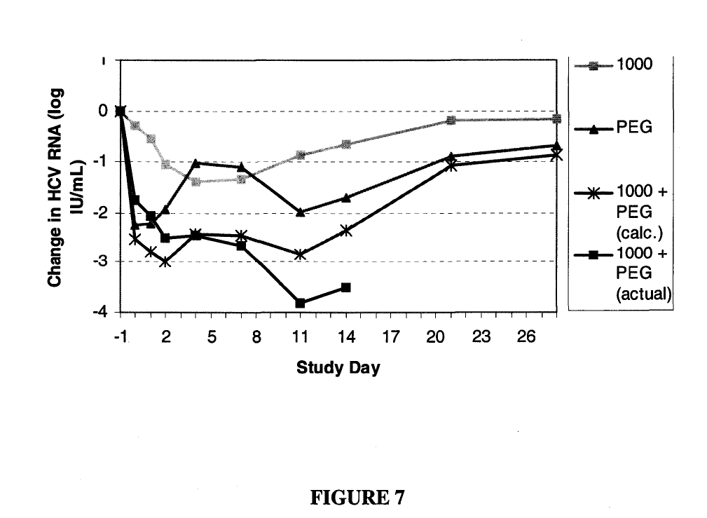

### DETAILED DESCRIPTION OF THE INVENTION

The present invention is based on drug development efforts which demonstrated that 5-cyclopropyl-2-(4-fluoro-phenyl)-6-[(2-hydroxy-ethyl)-methanesulfonyl-amino]-benzofuran-3-carboxylic acid methylamide and interferon, when administered as a combination therapy, produce significant reductions in virus levels in hepatitis C patients. The effectiveness of this combination therapy has been confirmed in a clinical study, the details of which are set forth in the examples that follow. Briefly, the combination therapy data described below demonstrate antiviral effects of 5-cyclopropyl-2-(4-fluoro-phenyl)-6-[(2-hydroxy-ethyl)-methanesulfonyl-amino]-benzofuran-3-carboxylic acid methylamide across multiple HCV genotypes, in treatment-naïve adult subjects with chronic hepatitis C infection. Across all dose groups, the combination of 5-cyclopropyl-2-(4-fluoro-phenyl)-6-[(2-hydroxy-ethyl)-methanesulfonyl-amino]-benzofuran-3-carboxylic acid methylamide and pegylated interferon produced a mean viral reduction of between 3.3 and 3.5 log 10 after 14 days of treatment, compared to 1.7 log 10 with pegylated interferon alone. There was no evidence of viral rebound over the dosing period relative to the effects of pegylated interferon alone. No dose-limiting toxicities were seen and although safety data remain blinded, tolerability was consistent with that expected from interferon treatment.

Based on these results, it is believed that 5-cyclopropyl-2-(4-fluoro-phenyl)-6-[(2-hydroxy-ethyl)-methanesulfonyl-amino]-benzofuran-3-carboxylic acid methylamide combined with interferon may significantly improve treatment outcomes compared to the current standard of care.

The term “INF” as used herein refers to “interferon” and “PEG-INF” as used herein refers to “pegylated interferon.” The term “HCV” as used herein refers to “hepatitis C virus.” The term “BZFs” as used herein refers to the benzofuran compounds described in WIPO publication WO 2004/041201 and the term “BZF” as used herein refers to “2-(4-fluoro-phenyl)-6-[(2-hydroxy-ethyl)-methanesulfonyl-amino]-benzofuran-3-carboxylic acid methylamide.”

In carrying out the combination therapy methods of the invention for treatment of HCV infections and diseases associated with such infections in a living host, a therapeutically effective amount of the BZF and a therapeutically effective amount of interferon are administered to a host susceptible to or suffering from such infection.

The term “combination therapy”, as used herein, refers to: (a) a therapy wherein two or more mono-therapies or active components in separate dosages are combined in a method of therapeutic treatment, and (b) a therapy wherein two or more therapeutic agents are formulated in a combined dosage which is administered in a method of therapeutic treatment.

The term “living host” as used herein refers to an organism that is living and capable of being infected with the hepatitis C virus, including mammals, particularly humans.

The combination therapy of this invention lowers the HCV-RNA by an additive-plus amount. The normal course of the therapy would be to continue the combination therapy for 1 to 100 days after an additive-plus effect is observed or would be until a therapeutic endpoint described below is reached. The expression “additive-plus amount” is used herein to signify an amount corresponding to an experimental result that is unexpectedly greater than the theoretical additive effect from combining two (or more) mono-therapies or active components (such as interferon and the above-mentioned benzofuran derivative). For example, if the theoretical additive effect of two active components or mono-therapies would be to reduce the HCV-RNA level by 50%, a reduction of 60% by the combination therapy would constitute an additive-plus amount of 1.2. The additive-plus amount in the practice of this invention is generally 1.2 or more times greater than the theoretical additive amount, preferably equal to or greater than 10, more preferably equal to or greater than 100 and most preferably equal to or greater than 1000.

The therapeutic efficacy of the combination therapy described herein may be measured in terms of a reduction of viral rebound during the course of therapy, as compared to a corresponding mono-therapy. The term “viral rebound” is used herein to refer to the resurgence in viral load in a living host during or after anti-viral therapy. The combination therapy is considered to have an added beneficial effect when a reduction in viral rebound observed during the course of therapy is maintained after cessation of the therapy. Ideally, the viral rebound will not only be reduced, but viral levels will be undetectable during the course of combination therapy, and after the therapy has stopped.

A desirable therapeutic end point of the combination therapy is an undetectable level of HCV-RNA during the therapy, which persists after cessation of the therapy. The term “BQL” as used herein refers to “below quantitative level.” A potential therapeutic endpoint for the combination therapy is when the HCV reduction in the living host is BQL for at least 10 days, preferably at least 30 days, more preferably at least 180 days, most preferably at least 300 days. Alternatively, a therapeutic endpoint for the combination therapy is when the HCV viral rebound is reduced or eliminated in the living host for at least 10 days, preferably at least 30 days, more preferably at least 180 days, most preferably at least 300 days. The appropriate methods of measuring and clinically quantitating HCV-RNA (and HCV infection) are known to one of ordinary skill in the art.

The combination therapy can be sequential, that is, treatment with one component of the basic combination of BZF and INF, followed by the other, at different times and/or at different frequencies or it can be practiced using the components of the basic combination simultaneously or concurrently. The treatment using both components at the same time can be in the same dosage or in separate dosages. The dosages for both concurrent and sequential combination therapy will depend on absorption, distribution, metabolism, and excretion rates of the components of the combination therapy as well as other factors known to one of skill in the art. Dosage values will also vary with the severity of the condition to be alleviated. It is to be further understood that for any particular subject, specific dosage regimens and schedules may be adjusted over time according to the individual's need and the professional judgment of the person administering or supervising the administration of the combination therapy. In practicing the methods of the invention, the first dose of interferon in the combination therapy is administered before the first dose of the 5-cyclopropyl-2-(4-fluoro-phenyl)-6-[(2-hydroxy-ethyl)-methanesulfonyl-amino]-benzofuran-3-carboxylic acid methylamide. Alternatively, the first dose of interferon in the combination therapy is administered after the first dose of the 5-cyclopropyl-2-(4-fluoro-phenyl)-6-[(2-hydroxy-ethyl)-methanesulfonyl-amino]-benzofuran-3-carboxylic acid methylamide. As another option, the first dose of interferon in the combination therapy is administered at the same time as the first dose of the 2-(4-fluoro-phenyl)-6-[(2-hydroxy-ethyl)-methanesulfonyl-amino]-benzofuran-3-carboxylic acid methylamide.

The combination therapy described herein may also be practiced using other biologically active agents, including but not limited to the group consisting of ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds. This modified combination therapy may also involve administering the basic combination of the benzofuran derivative and interferon, either concurrently or sequentially, with other medicinal agents or potentiators, such as acyclovir, famciclovir, valganciclovir or related compounds.

In a further embodiment, the basic combination of benzofuran derivative and interferon may be used for the treatment of HCV in humans in a modified combination therapy, including other inhibitors of the HCV polymerase.

In yet a further embodiment, the basic two-component combination described herein may be used for the treatment of HCV in humans in a modified combination therapy, together with other inhibitors of the HCV life cycle such as, for example, inhibitors of HCV cell attachment or virus entry, HCV translation, HCV RNA transcription or replication, HCV maturation, assembly or virus release, or inhibitors of HCV enzyme activities such as the HCV nucleotidyl transferase, helicase, protease or polymerase.

It is intended that combination therapies contemplated to be within the scope of this invention include any chemically compatible combination of a 5-cyclopropyl-2-(4-fluoro-phenyl)-6-[(2-hydroxy-ethyl)-methanesulfonyl-amino]-benzofuran-3-carboxylic acid methylamide, interferon and, optionally, another therapeutic agent, as long as the resultant combination does not adversely affect the anti-viral activity of the basic two components.

The interferon component of this invention may be in various forms, including, without limitation, interferon-alpha, interferon-beta, interferon-gamma, and the like, as well as alternative form of interferons, such as pegylated interferons. The term “interferon” as used herein means and includes all of these forms.

The term “interferon-alpha” as used herein means the family of highly homologous species-specific proteins that inhibit viral replication and cellular proliferation and modulate immune response. Typical suitable interferon-alphas include, but are not limited to, recombinant interferon alpha-2b such as INTRON-A INTERFERON available from Schering Corporation, Kenilworth, N.J., recombinant interferon alpha-2a such as Roferon interferon available from Hoffmann-La Roche, Nutley, N.J., a recombinant interferon alpha-2C, such as BEROFOR ALPHA 2 INTERFERON available from Boehringer Ingelheim Pharmaceutical, Inc., Ridgefield, Conn., interferon alpha-n1, a purified blend of natural alpha interferons such as SUMIFERON available from Sumitomo, Japan or as Wellferon interferon alpha-n1 (INS) available from Glaxo-Wellcome Ltd., London, Great Britain, or a consensus alpha interferon such as those described in U.S. Pat. Nos. 4,897,471 and 4,695,623 (the contents of which are hereby incorporated by reference in their entireties, specifically examples 7, 8 or 9 thereof) and the specific product available from Amgen, Inc., Newbury Park, Calif., or interferon alpha-n3 a mixture of natural interferons made by Interferon Sciences and available from the Purdue Frederick Co., Norwalk, Conn., under the ALFERON trademark. The use of interferon alpha-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,503,901, the entire disclosure of which is incorporated by reference herein.

The term “pegylated interferon” as used herein means polyethylene glycol modified conjugates of interferon, preferably interferon alpha-2a and interferon alpha-2b. The preferred polyethylene-glycol-interferon alpha-2b conjugate is PEG.sub.12000-interferon alpha 2b. The phrase “PEG.sub.12000-IFN alpha” as used herein means conjugates such as are prepared according to the methods of International Application No. WO 95/13090 and containing urethane linkages between the interferon alpha-2a or alpha-2b amino groups and polyethylene glycol having an average molecular weight of 12000.

The BZF used in the practice of this invention can form useful salts with inorganic and organic acids such as hydrochloric, sulfuric, acetic, lactic, or the like and with inorganic or organic bases such as sodium or potassium hydroxide, piperidine, ammonium hydroxide, or the like. Such pharmaceutically acceptable salts are prepared following procedures that are familiar to those skilled in the art. For example, sodium and potassium salts can be made by dissolving the benzofuran derivative in ethanol and adding about 1.1 equivalents of sodium hydroxide or potassium hydroxide, and allowing salt formation.

The BZF may also be used in its corresponding possible tautomeric forms. Such tautomers may, in certain instances, be resolved into individual compounds by methods known to those of skill in the art.

The combination therapy method may be carried out using the above-mentioned BZF, or pharmaceutically acceptable salts thereof, together with interferon, and a pharmaceutically acceptable carrier medium formulated in a combined dosage. As used herein, “pharmaceutically acceptable carrier medium” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Twentieth Edition, A. R. Gennaro (William and Wilkins, Baltimore, Md., 2000) discloses various carriers used in formulating pharmaceutical compositions and known techniques for the preparation thereof. Except insofar as any conventional carrier medium is incompatible with the antiviral compounds used to practice this invention, such as by producing any undesirable biological effect or otherwise interacting in a deleterious manner with any other component(s) of the pharmaceutical composition, its use is contemplated to be within the scope of this invention.

Pharmaceutical organic or inorganic solid or liquid carrier media suitable for enteral or parenteral administration can be used to make up the dosages. Gelatine, lactose, starch, magnesium stearate, talc, vegetable and animal fats and oils, gum, polyalkylene glycol, or other known medicament components may all be suitable as carrier media or excipients.

The therapeutic agents used in practicing this invention may be administered using any amount and any route of administration effective for inhibiting viral replication and attenuating HCV infectivity. The expression “amount effective to inhibit viral replication,” as used herein, refers to a nontoxic but sufficient amount of the composition(s) to provide the desired treatment of HCV infection, preferably an additive-plus effect (preferably) and/or a reduction in viral rebound (more preferably) during or after administration of the combination therapy. The exact amount required will vary from subject to subject, depending on the species, age, and general condition of the subject, the severity of the infection, the particular antiviral agent, its mode of administration, and the like. The therapeutic agents may be given in the following amounts: BZF (50-10,000 mg/day); PEG-INF (5-250 μg/dose every 3 to 14 days). In general, interferon amounts that may be used in this invention are the amounts conventionally used by one of ordinary skill in the art for antiviral therapy, and particularly for treating hepatitis C infections.

The separate dosages of the basic components used for combination therapy, or the combined dosage (when used), are preferably formulated in dosage unit form for ease of administration and uniformity of dosage. “Dosage unit form” as used herein refers to a physically discrete unit of antiviral agent appropriate for the patient to be treated. Each dosage should contain the quantity of active material calculated to produce the desired therapeutic effect either as such, or in association with the selected pharmaceutical carrier medium and/or the supplemental active agent(s), if any. Typically, the BZF will be administered in dosage units containing from about 25 mg to about 1500 mg of the active agent, with a range of about 250 mg to about 500 mg being preferred. The IF will be administered in dosage unit containing from about 0.5 μg/kg to about 2.5 μg/kg of the active agent, with a range of about 1.0 μg/kg to about 1.5 μg/kg being preferred. A combined dosage will typically include from about 25 mg to about 1000 mg of the BZF, and from about 0.5 μg/kg to about 2.0 μg/kg of INF, with about 250 mg to about 500 mg BZF and from about 1.0 μg/kg to about 1.5 μg/kg INF, respectively, being preferred.

The components of the therapeutic combination may be prepared in various forms for administration, including tablets, caplets, pills or dragees, or can be filled in suitable containers, such as capsules, or, in the case of suspensions, filled into bottles.

The therapeutic agents may be administered orally, rectally, parenterally, such as by intramuscular injection, subcutaneous injection, intravenous infusion or the like, intracisternally, intravaginally, intraperitoneally, locally, such as by powders, ointments, or drops, or the like, or by inhalation, such as by aerosol or the like, taking into account the nature and severity of the infection being treated. The BZF and INF need not be, and typically are not delivered by the same route of administration. Depending on the route of administration, the therapeutic agent may be administered at dosage levels of about 1.0 mg to 100 mg/kg of subject body weight per day, one or more times a day in the case of the BZF, and about 0.1 to 3.0 μg/kg of subject body weight per day, in the case of the PEG-INF, in one or more doses per day to obtain the desired therapeutic effect. These dosage levels assume an average weight of an adult male to be about 80 kg, and an average weight of an adult female to be about 55 kg.

One or more of the therapeutic agents of the invention will typically be administered from 1 to 3 times a day so as to deliver the above-mentioned daily dosage. However, the exact regimen for administration of the compositions described herein will necessarily be dependent on the needs of the individual host or patient being treated, the type of treatment administered and the judgment of the attending medical specialist. For example, benzofuran derivative and/or the interferon may be administered by continuous infusion or via a controlled release formulation.

The BZF and interferon may be conveniently packaged as an article of manufacture for administration of the combination therapy, which comprises, for example, a vial containing the BZF component, or a pharmaceutically acceptable salt, with a pharmaceutically acceptable carrier, and a vial containing the INF component with a pharmaceutically acceptable carrier, in a form suitable for co-administration, either simultaneously or sequentially, including administration of BZF and INF at unequal time intervals.

In view of the inhibitory effect on viral RNA synthesis produced by the combination therapy of the invention, it is anticipated that these compounds will be useful not only for therapeutic treatment of virus infection, but for virus infection prophylaxis in susceptible subjects, as well. The dosages may be essentially the same, whether for treatment or prophylaxis of virus infection.

The combinations of antiviral agents described herein may also be useful in preventing or resolving viral infections in cell, tissue or organ cultures and other in vitro applications. For example, inclusion of compositions of the invention as a supplement in cell or tissue culture growth media and cell or tissue culture components will prevent viral infections or contaminations of cultures not previously infected with viruses. The compositions described herein may also be used to eliminate viruses from cultures or other biological materials infected or contaminated with viruses (for example, blood), after a suitable treatment period, under any number of treatment conditions as determined by the skilled artisan.

The use of the combination therapy additionally may result in synergism between the combined components, and/or result in synergist therapeutic effects and benefits.

The following examples are provided to describe the invention in further detail. These examples include a suitable method of synthesis of the benzofuran derivative used in this invention. However, this method of synthesis is intended merely to illustrate and not to limit the invention. The starting materials for preparing the exemplified benzofuran derivative are either commercially available or can be conveniently prepared according to one of the examples set forth below or otherwise using known chemistry procedures. The term “HPLC,” as used herein, refers to high-performance liquid chromatography. The term “psig” refers to pounds per square inch gauge.

### EXAMPLE 1

### Preparation of 5-cyclopropyl-2-(4-fluoro-phenyl)-6-[(2-hydroxy-ethyl)-methanesulfonyl-amino]-benzofuran-3-carboxylic acid methylamide

a. Preparation of cyclopropylboronic acid. Cyclopropylboronic acid was prepared from cyclopropylmagnesium bromide, according to the literature procedure: Wallace, D. J., Chen, C., Tetrahedron Lea. 2002, 43, 6987-6990, on a 4 g scale (56% yield).

b. Preparation of 2-(4-fluoro-phenyl)-5-hydroxy-6-nitro-benzofuran-3-carboxylic acid ethyl ester. Boron trichloride (106 mL, 0.106 mol) was added to dropwise to a solution of compound 21(d) (20.5 g, 0.053 mol, which can be prepared according to Example 21, steps a-d, above) in anhydrous dichloromethane (264 mL), under argon. The reaction mixture was stirred at ambient temperature overnight. The reaction was quenched with ice water and extracted with dichloromethane (3×). The organic layers were combined, dried over magnesium sulfate, filtered, and evaporated. The resulting solid was sonicated in hexanes, filtered and dried to provide 17.82 g (99%) of the product as a yellow solid.

c. Preparation of 2-(4-fluoro-phenyl)-6-nitro-5-trifluoromethanesulfonyloxy-benzofuran-3-carboxylic acid ethyl ester. N,N-Diisopropylethylamine (8.8 mL, 56 mmol) and 4-(dimethylamino)pyridine (0.618 g, 5.06 mmol) were added to a suspension of the compound prepared according to step a above, in anhydrous dichloromethane (300 mL) under argon. The reaction mixture was cooled to 0° C. in an ice/water bath, and then trifluoromethanesulfonic anhydride (9.34 mL, 56 mmol) was added. The reaction was stirred at ambient temperature for about 5 hours, and then additional amounts of N,N-diisopropylethylamine (4.4 mL, 28 mmol) and trifluoromethanesulfonic anhydride (4.67 mL, 28 mmol) were added. The reaction was stirred at room temperature overnight, diluted with water and extracted with dichloromethane (3×). The organic layers were washed with water (3×) and 1N HCl (1×), combined, dried over magnesium sulfate, and evaporated. The residue was recrystallized from t-butylmethyl ether to provide a total of 20.36 g (84%) of the desired product as a yellow solid.

d. Preparation of 5-cyclopropyl-2-(4-fluoro-phenyl)-6-nitro-benzofuran-3-carboxylic acid ethyl ester. Anhydrous toluene (10.0 mL) was added to a mixture of cyclopropylboronic acid (0.271 g, 3.14 mmol), potassium fluoride dihydrate (0.652 g, 6.92 mmol), sodium bromide (0.216 g, 2.16 mmol), tetrakis(triphenylphosphine)palladium(0) (0.073 g, 0.0629 mmol), and compound 43(b) (1.0 g, 2.09 mmol). The resulting solution was degassed with argon through a gas dispersion tube for 10 minutes. The reaction mixture was heated to reflux overnight, diluted with water, and extracted with ethyl acetate (3×). The organic layers were combined, dried over magnesium sulfate, and evaporated. The crude product was purified by column chromatography (silica gel, dry loading, hexane/ethyl acetate gradient) to afford 0.670 g (86%) of the desired product as a solid.

e. Preparation of 6-amino-5-cyclopropyl-2-(4-fluoro-phenyl)-benzofuran-3-carboxylic acid ethyl ester. 10% Palladium on carbon (0.150 g) and 1N HCl (7 drops) were added to a solution of compound 43(c) (0.665 g, 1.8 mmol) in ethyl acetate (70.0 mL). The reaction mixture was shaken under 50 psig of hydrogen gas on a Parr shaker overnight. The reaction mixture was filtered through Celite™, rinsing with ethyl acetate and methanol. The filtrate was concentrated in vacuo to afford 0.540 g (88%) of the desired product as a solid.

f. Preparation of 5-cyclopropyl-2-(4-fluoro-phenyl)-6-methanesulfonylamino-benzofuran-3-carboxylic acid. Methanesulfonylchloride (0.270 mL, 3.48 mmol) was added to chilled solution (0° C., ice/water bath) of compound 43(d) (0.535 g, 1.58 mmol) dissolved in dichloromethane (6 mL). The reaction mixture was cooled further in an ethanol/ice bath, and then N,N-diisopropylethylamine (0.688 mL, 3.95 mmol) was added. The reaction was stirred at room temperature overnight, diluted with water, and extracted with dichloromethane (3×). The organic layers were combined, dried over magnesium sulfate, and evaporated to afford 0.653 g (86%) of the bis(sulfonylated) intermediate.

Potassium hydroxide (1.52 g, 27 mmol) was added to a solution of the bis(sulfonylated) intermediate (0.670 g, 1.35 mmol) dissolved in ethanol (10.0 mL) and water (5.0 mL) under argon. The reaction was heated to reflux overnight, and then concentrated in vacuo. The remaining solid was dissolved in water, and the solution was acidified with 1N HCL until a precipitate formed. The solid was filtered and dried to afford 0.532 g (99%) of the desired product.

g. Preparation of 5-cyclopropyl-2-(4-fluoro-phenyl)-6-methanesulfonylamino-benzofuran-3-carboxylic acid methylamide. Benzotriazol-1-yloxytrispyrrolidinophosphonium hexafluorophosphate (PyBop) (1.02 g, 1.97 mmol) was added to a mixture of methylamine (12.0 mL, 16.3 mmol, 2.0M in THF), DMF (1.0 mL), and compound 43(e) (0.530 g, 1.36 mmol) under argon. The reaction was stirred at room temperature overnight and then concentrated in vacuo. The residue was diluted with water and extracted with ethyl acetate (3×). The organic layers were combined, washed with water, dried over magnesium sulfate, and air-dried to afford 0.347 g (63%) of crude product. A portion of the crude product (100 mg) was purified by reverse phase HPLC (acetonitrile/water gradient) to afford 0.050 g of the desired product.

h. Preparation of 6-[(2-benzyloxy-ethyl)-methanesulfonyl-amino]-5-cyclopropyl-2-(4-fluoro-phenyl)-benzofuran-3-carboxylic acid methylamide. Potassium carbonate (0.125 g, 0.91 mmol) and benzyl 2-bromoethyl ether (0.105 mL, 0.67 mol) were added to a solution of the compound prepared in step g., above, (0.120 g, 0.0003 mol), dissolved in acetonitrile (1.5 mL) under argon. The reaction mixture was heated to reflux overnight, diluted with water, and extracted with dichloromethane (3×). The organic layers were combined, dried over magnesium sulfate, and evaporated. The crude product was purified by reprecipitating out of ethyl acetate/hexanes, and the isolated solid was taken on to the next step without further purification.

i. Preparation of title compound. 10% Palladium on carbon (0.100 g) was added to mixture of the compound prepared in step h., above, (0.162 g, 0.41 mmol) in ethyl acetate (20 mL). The reaction mixture was shaken under 50 psig of hydrogen gas on a Parr shaker overnight. The reaction mixture was filtered through Celite™, rinsing with ethyl acetate and methanol. The filtrate was concentrated in vacuo, and the crude product was dissolved in ethyl acetate and precipitated with hexanes. The solid was isolated by filtration to afford 0.083 g of desired product as a tan solid.

NMR data: ¹H NMR in CDCl₃: 7.90-7.85 (m, 2H); 7.55 (s, 1H); 7.35 (s, 1H); 7.22-7.16 (m, 2H); 5.74 (brs, 1H); 4.09-4.03 (m, 1H); 3.75 (s, 3H); 3.14 (s, 3H); 2.99 (d, J=4.40 Hz, 3H); 2.40-2.30 (m, 1H); 1.95 (m, 1H); 1.10-0.98 (m, 2H); 0.88 (m, 1H); 0.68 (m, 1H); mass spec. data: (M+H)⁺=447.

In the foregoing example, NMR spectra were acquired on a Varian Mercury VX 300 Spectrometer and referenced to tetramethylsilane (TMS. Chemical shifts and coupling constants are reported in parts per million (ppm) and Hertz (Hz) respectively. Multiplicities indicated are: s=singlet, d=doublet. m=multiplet, and br indicates a broad signal. Mass spectroscopy data is expressed as a mass to charge ratio (m/z).

### EXAMPLE 2

### Inhibition of Viral RNA Replication

Antiviral activity of the BZF of Example 1 was first evaluated in a human liver-derived cell line (Huh-7-Clone A) containing the HCV replicon (BB7 sequence) (See Lohmann et al. Science. 1999, 285:110-3; Blight K J et al., Science. 2000, 290:1972-4; Pietschmann, T. et al., J. Virol. 2001, 73:1252-1264; and Lohmann, V. et al., J. Virol. 2001, 75:1437-1449). The HCV replicon is a subgenomic viral RNA that expresses the HCV proteins required for its own replication. These proteins include non-structural proteins NS3, NS4A, NS4B, NS5A and NS5B. The replicon also contains a foreign gene encoding a drug-selectable marker (neomycin phosphotransferase) to allow for G418 (neomycin) selection of cells that contain the replicon.

The antiviral activity of the BZF of Example 1 was evaluated in the human hepatoma cells (Huh-7 cells) containing a genotype 1b (BB7 isolate) HCV replicon. The cells were treated with increasing concentrations of compounds in medium containing 2% FCS and no G418 for three days at 37° C. and 5% CO₂. After 3 days of incubation, total RNA from the replicon-containing cells was isolated. The levels of HCV, glyceraldehyde 3-phosphate dehydrogenase (GAPDH), and ribosomal (rRNA) RNAs were quantified using a fluorescence-based quantitative real time polymerase chain reaction (RT-PCR) assay, also known as TaqMan® RT-PCR, that determined the steady-state levels of HCV RNA in the cells. The amounts of HCV, 18S ribosomal, and GAPDH RNAs in each sample were estimated by comparing the number of cycles during the exponential phase of the PCR amplification with those in the corresponding standard curves. HCV RNA standards used for the construction of the standard curve were prepared by extracting the total RNA from Clone A cells. The RNA sample was sent to National Genetics Institute to quantify HCV RNA. Total RNA extracted from Clone A cells was quantified by O.D.₂₆₀ measurement and used for construction of the standard curves of rRNA and GAPDH. The concentrations of the compounds that inhibit 50% of the HCV RNA level (EC₅₀) were determined using the MDL LSW Data Analysis™ software in Microsoft Excel™. The amounts of HCV or GAPDH RNAs in the samples were expressed as HCV RNA (copies) or GAPDH (ng), respectively, per μg of total RNA using rRNA as a marker for total RNA measurement. The BZF showed a dose-dependent inhibition of intracellular HCV RNA levels. The EC₅₀ values are less than 100 mM. For combination studies, the resulting data were analyzed using the Bliss Independence null model of additivity (MacSynergy™ II).

### EXAMPLE 3

### Inhibition of Viral RNA-Dependent RNA Polymerase (RdRp)

The HCV NS5B-directed RdRp activity was established and characterized in a standard in vitro biochemical assay using a purified HCV NS5B protein derived from the consensus sequence of a patient infected with HCV genotype 1b virus (BB7). (See Blight K J et al., Science. 2000, 290:1972-4). The NS5B consensus sequence was cloned and expressed in E. coli as a histidine tagged (GSHHHHHH) fusion protein, of which the carboxyl terminal 21 amino acids were removed to enhance its solubility.

In addition to evaluating its activity in the replicon assay, as described above, the BZF of Example 1 was also evaluated for antiviral activity using this assay. A measure of the inhibitory activity of the compounds may be expressed as an IC50 value, which represents the concentration of the compound at which 50% of the RdRp activity (IC₅₀) is inhibited. The result of the assay for inhibition of RdRp activity of HCV, NS5B proteins for the compound tested revealed an IC₅₀ value of <0.5 μM. This low concentration of test compounds required to achieve 50% inhibition of the RdRp activity indicates that the compound is effective at inhibiting RNA synthesis by viral RdRp enzymes.

### EXAMPLE 4

### Phase 1b Clinical Trial Description

A 14 day randomized, double-blind, placebo-controlled, sequential-group study of multiple ascending doses (MAD) included subjects with chronic HCV infection who were naïve to treatment. Subjects were enrolled in sequential, ascending dose cohorts with a target of 16 subjects (12 subjects receiving the BZF and 4 receiving placebo in each cohort). The first cohorts assessed the effect of the BZF as monotherapy compared to placebo. Subsequent cohorts were comprised of subjects who received pegylated interferon alfa-2b (PEG-Intron; 1.5 μg/kg/dose) administered subcutaneously on days −1 and 7 in combination with either placebo or the BZF administered orally (100 mg, 250 mg, 500 mg or 1000 mg every 12 hours, in combinations of 25 mg or 200 mg capsules) from days 1 to 14. Sixty-four percent of patients in the combination therapy cohorts were infected with HCV genotype 1. The mean viral load for each treatment group at study entry was >6.0 log 10 UI/ml HCV RNA.

The BZF capsules used in the Phase 1b clinical trial were formulated as follows:

Active Ingredient:

BZF (25 mg, or 200 mg)

Inactive Ingredients

Microcrystalline Cellulose

Polysorbate 80 (vegetable grade)

Povidone

Sodium Starch Glycolate

Sodium Lauryl Sulfate

Silicon Dioxide, Colloidal

Magnesium Stearate (vegetable grade)

#OE HPMC (Hydroxypropyl Methylcellulose, or Hypromellose) Capsule, Opaque Brown

The resultant capsules should be stored at 25° C. (771 F) or below; do not freeze. Excursions permitted to 30° C. (86° F.).

### Phase 1b Preliminary Clinical Results

Preliminary data are available through treatment day 14 from subjects in four combination treatment groups (n=9-11 subjects per group) and on 15 subjects who received pegylated interferon alone.
Across all combination groups, the mean reduction from baseline in plasma HCV RNA ranged from 2.1 to 2.7 log 10 on day 7 and 3.3 to 3.5 log 10 on day 14. This compared to a reduction of 1.1 log 10 on day 7 and 1.7 log 10 on day 14 with pegylated interferon alone.
   Consistent with known effects of pegylated interferon, response varies by HCV genotype. For genotype 1, mean reduction from baseline at day 14 ranged from 2.6 to 3.2 log 10 in the combination therapy groups versus 1.3 log 10 for pegylated interferon alone.
Viral reduction greater or equal to 2 log 10 at day 14 was achieved in 70 to 90 percent of subjects in all combination groups compared to 43% on pegylated interferon alone.
At day 14, 30 to 33 percent of patients in the combination group receiving ≧250 mg of BZF achieved viral levels below the limits of quantification.

Although safety data remained blinded, observed adverse events appear consistent with those expected with pegylated interferon, including common occurrence of flu-like symptoms such as headache, fever or chills.

TABLE 1
BZF: Change from Baseline (log HCV RNA level) - On-therapy
Analysis (MAD Study, All Genotypes)

| | Day 4 | | Day 7 | | Day 14 | |
|---|---|---|---|---|---|---|
| | N | MEAN Δ | N | MEAN Δ | N | MEAN Δ |
| 500 mg | 14 | −1.43 | 13 | −1.30 | 12 | −0.64 |
| PEG-INF | 15 | −1.03 | 14 | −1.11 | 14 | −1.69 |
| 100 mg + PEG-INF | 11 | −2.20 | 10 | −2.12 | 10 | −3.31 |
| 250 mg + PEG-INF | 9 | −2.55 | 9 | −2.47 | 9 | −3.31 |
| 500 mg + PEG-INF | 10 | −2.58 | 10 | −2.62 | 10 | −3.33 |
| 1000 mg + PEG-INF | 11 | −2.46 | 10 | −2.66 | 10 | −3.51 |

TABLE 2
BZF: MAD Study, Combo Cohorts - Baseline HCV RNA

Baseline log
HCV RNA level

| Dose Group | n | mean | range |
|---|---|---|---|
| 100 mg + PEG-INF | 11 | 6.41 | 5.04-7.29 |
| 250 mg + PEG-INF | 9 | 6.15 | 4.65-7.02 |
| 500 mg + PEG-INF | 10 | 6.45 | 5.67-7.22 |
| 1000 mg + PEG-INF | 11 | 6.50 | 5.64-7.38 |
| PEG-INF ONLY | 15 | 6.59 | 5.80-7.14 |
| Total | 56 | 6.44 | 4.65-7.38 |

Overall, 80% had baseline HCV RNA level ≧6 log

TABLE 3
HCV-RNA Reductions - all BZF doses
(Change from Baseline at Day 14, All Genotypes)
% Subjects With Various ↓ in log10 HCV RNA, on-therapy analysis

| | n | ≧1.5 log | ≧2 log | ≧3 log | BQL |
|---|---|---|---|---|---|
| 500 mg | 12 | 1 (8%) | 1 (8%) | 0 | 0 |
| PEG-INF | 14 | 7 (50%) | 6 (43%) | 2 (14%) | 2 (14%) |
| 100 mg + PEG-INF | 10 | 9 (90%) | 7 (70%) | 7 (70%) | 2 (20%) |
| 250 mg + PEG-INF | 9 | 8 (89%) | 7 (78%) | 6 (67%) | 3 (33%) |
| 500 mg + PEG-INF | 10 | 10 (100%) | 9 (90%) | 6 (60%) | 3 (30%) |
| 1000 mg + PEG-INF | 10 | 9 (90%) | 7 (70%) | 7 (70%) | 3 (30%) |

BQL = below quantification limits (<50 IU/mL)

TABLE 4
HCV-RNA Reductions - 100 mg BZF/PEG-INF
(Change from Baseline at Day 14, All Genotypes)
% Subjects With Various ↓ in log10 HCV RNA, on-therapy analysis

| | n | ≧1.5 log | ≧2 log | ≧3 log | BQL |
|---|---|---|---|---|---|
| 100 mg | 12 | 2 (17%) | 0 | 0 | 0 |
| PEG-INF | 14 | 7 (50%) | 6 (43%) | 2 (14%) | 2 (14%) |
| 100 mg + PEG-INF | 10 | 9 (90%) | 7 (70%) | 7 (70%) | 2 (20%) |

BQL = below quantification limits (<50 IU/mL)

TABLE 5
HCV-RNA Reductions - 250 mg BZF/PEG-INF
(Change from Baseline at Day 14, All Genotypes)
% Subjects With Various ↓ in log10 HCV RNA, on-therapy analysis

| | n | ≧1.5 log | ≧2 log | ≧3 log | BQL |
|---|---|---|---|---|---|
| 250 mg | 12 | 0 | 0 | 0 | 0 |
| PEG-INF | 14 | 7 (50%) | 6 (43%) | 2 (14%) | 2 (14%) |
| 250 mg + PEG-INF | 9 | 8 (89%) | 7 (78%) | 6 (67%) | 3 (33%) |

BQL = below quantification limits (<50 IU/mL)

TABLE 6
HCV-RNA Reductions - 500 mg BZF/PEG-INF
(Change from Baseline at Day 14, All Genotypes)
% Subjects With Various ↓ in log10 HCV RNA, on-therapy analysis

| | n | ≧1.5 log | ≧2 log | ≧3 log | BQL |
|---|---|---|---|---|---|
| 500 mg | 12 | 1 (8%) | 1 (8%) | 0 | 0 |
| PEG-INF | 14 | 7 (50%) | 6 (43%) | 2 (14%) | 2 (14%) |
| 500 mg + PEG-INF | 10 | 10 (100%) | 9 (90%) | 6 (60%) | 3 (30%) |

BQL = below quantification limits (<50 IU/mL)

TABLE 7
HCV-RNA Reductions - 1000 mg BZF/PEG-INF
(Change from Baseline at Day 14, All Genotypes)
% Subjects With Various ↓ in log10 HCV RNA, on-therapy analysis

| | n | ≧1.5 log | ≧2 log | ≧3 log | BQL |
|---|---|---|---|---|---|
| 1000 mg | 12 | 2 (17%) | 2 (17%) | 1 (8%) | 0 |
| PEG-INF | 14 | 7 (50%) | 6 (43%) | 2 (14%) | 2 (14%) |
| 1000 mg + PEG-INF | 10 | 9 (90%) | 7 (70%) | 7 (70%) | 3 (30%) |

BQL = below quantification limits (<50 IU/mL)

TABLE 8
HCV-RNA Reductions - 500 mg BZF/PEG-INF
(Change from Baseline at Day 14, All Genotype 1)
% Subjects With Various ↓ in log10 HCV RNA, on-therapy analysis

| | n | ≧1.5 log | ≧2 log | ≧3 log | BQL |
|---|---|---|---|---|---|
| 500 mg | 8 | 1 (13%) | 1 (13%) | 0 | 0 |
| PEG-INF | 10 | 4 (40%) | 4 (40%) | 1 (10%) | 1 (10%) |
| 500 mg + PEG-INF | 6 | 6 (100%) | 5 (83%) | 2 (33%) | 0 |

BQL = below quantification limits (<50 IU/mL)

While not wishing to be bound to a particular theory, it is believed that the clinical trial data presented in this example tends to show that the combination of BZF and PEG-INF produces a synergistic therapeutic effect against HCV and/or an additive-plus effect. The expression “synergistic therapeutic effect,” as used herein refers to an effect that arises from administration of BZF and PEG-INF in combination which is greater than the algebraic sum of the effects resulting from the separate administration of BZF and PEG-INF, in accordance with established principles of synergism theory that are known to those of ordinary skill in the art.

Although the present invention has been described and exemplified in terms of certain preferred embodiments, other embodiments will be apparent to those skilled in the art. The invention is, therefore, not limited to the particular embodiments described and exemplified, but is capable of modification or variation without departing from the spirit of the invention, the full scope of which is delineated by the appended claims.

What is claimed is:

1. A method of treating a living host having hepatitis C virus infection using a combination therapy comprising administering a therapeutically effective amount of a 5-cyclopropyl-2-(4-fluorophenyl)-6-[(2-hydroxy-ethyl)-methanesulfonyl-amino]-benzofuran-3-carboxylic acid methylamide and a therapeutically effective amount of interferon for a time period sufficient to lower the HCV-RNA in the living host by an additive-plus amount.

2. The method according to claim 1, wherein said additive-plus amount is equal to or greater than 1.2 times the theoretical additive amount of HCV-RNA level reduction.

3. The method according to claim 1, wherein said additive-plus amount is equal to or greater than 10 times the theoretical additive amount of HCV-RNA level reduction.

4. The method according to claim 1, wherein said additive-plus amount is equal to or greater than 100 times the theoretical additive amount of HCV-RNA level reduction.

5. The method according to claim 1, wherein said additive-plus amount is equal to or greater than 1000 times the theoretical additive amount of HCV-RNA level reduction.

6. The method according to claim 1, wherein said method lowers the HCV-RNA to an undetectable level during or after the combination therapy.

7. The method according to claim 1, wherein said combination therapy produces a measurable synergistic therapeutic effect on the HCV-RNA level.

8. The method of claim 1, wherein said interferon is selected from the group consisting of interferon-alpha, interferon-beta, interferon-gamma and pegylated interferon.

9. The method of claim 8, wherein said interferon is interferon-alpha.

10. The method of claim 1, wherein said interferon is pegylated interferon-alpha-2b.

11. The method according to claim 1 further comprising the step of administering at least one supplemental biologically active agent selected from the group of ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

12. The method according to claim 1, wherein said 5-cyclopropyl-2-(4-fluoro-phenyl)-6-[(2-hydroxy-ethyl)-methanesulfonyl-amino]-benzofuran-3-carboxylic acid methylamide and interferon are administered concurrently, in separate dosages.

13. The method according to claim 1, wherein the 5-cyclopropyl-2-(4-fluoro-phenyl)-6-[(2-hydroxy-ethyl)-methanesulfonyl-amino]-benzofuran-3-carboxylic acid methylamide and interferon are administered concurrently as a combined dosage.

14. The method according to claim 13, wherein the combined dosage composition further comprises at least one supplemental biologically active agent selected from the group consisting of ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

15. The method according to claim 1, wherein said living host is a mammal.

16. The method according to claim 15, wherein said living host is a human.

17. The method according to claim 1, wherein the 5-cyclopropyl-2-(4-fluoro-phenyl)-6-[(2-hydroxy-ethyl)-methanesulfonyl-amino]-benzofuran-3-carboxylic acid methylamide is administered orally.

18. The method according to claim 1, wherein the 5-cyclopropyl-2-(4-fluoro-phenyl)-6-[(2-hydroxy-ethyl)-methanesulfonyl-amino]-benzofuran-3-carboxylic acid methylamide is administered orally at a dose range of about 25 mg to 10,000 mg per day.

19. The method according to claim 18, wherein the 5-cyclopropyl-2-(4-fluoro-phenyl)-6-[(2-hydroxy-ethyl)-methanesulfonyl-amino]-benzofuran-3-carboxylic acid methylamide is administered from 1 to 3 times daily.

20. The method according to claim 1, wherein the 5-cyclopropyl-2-(4-fluoro-phenyl)-6-[(2-hydroxy-ethyl)-methanesulfonyl-amino]-benzofuran-3-carboxylic acid methylamide and interferon are administered either concurrently or sequentially, with at least one other biologically active agent.

21. The method according to claim 20, wherein said other biologically active agent is selected from the group consisting of ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

22. A method of treating a living host having hepatitis C infection using a combination therapy comprising administering a therapeutically effective amount of a 5-cyclopropyl-2-(4-fluoro-phenyl)-6-[(2-hydroxy-ethyl)-methanesulfonyl-amino]-benzofuran-3-carboxylic acid methylamide and a therapeutically effective amount of interferon for a time period sufficient to reduce viral rebound as compared to a corresponding 5-cyclopropyl-2-(4-fluoro-phenyl)-6-[(2-hydroxyethyl)-methanesulfonyl-amino]-benzofuran-3-carboxylic acid methylamide mono-therapy.

23. The method according to claim 22, wherein the viral rebound is reduced during said combination therapy.

24. The method according to claim 22, wherein the viral rebound is reduced after said combination therapy has stopped.

25. The method according to claim 22, wherein there is no detectable viral rebound during combination therapy.

26. The method according to claim 22, wherein there is no detectable viral rebound after combination therapy has stopped.

27. A method according to claim 22, wherein the combination therapy reduces or prevents pre-existing or emergent viral variants.

28. A method according to claim 22, wherein the viral rebound is due to pre-existing or emergent strains of hepatitis C virus that are resistant to 5-cyclopropyl-2-(4-fluoro-phenyl)-6-[(2-hydroxy-ethyl)methanesulfonyl-amino]-benzofuran-3-carboxylic acid methylamide treatment.

29. A method according to claim 22, wherein the combination therapy inhibits the emergence of hepatitis C virus that is less susceptible to 5-cyclopropyl-2-(4-fluoro-phenyl)-6-[(2-hydroxy-ethyl)-methanesulfonyl-amino]-benzofuran-3-carboxylic acid methylamide treatment.

30. An improved method of treating a living host having hepatitis C infection with interferon therapy comprising administering a therapeutically effective amount of a 5-cyclopropyl-2-(4-fluorophenyl)-6-[(2-hydroxy-ethyl)-methanesulfonyl-amino]-benzofuran-3-carboxylic acid methylamide in a combination therapy with a therapeutically effective amount of interferon for a time period sufficient to shorten the duration of treatment relative to a course of interferon mono-therapy or a course of interferon and ribavirin combination therapy.

31. A method of treating a living host having hepatitis C virus infection using a combination therapy comprising administering a therapeutically effective amount of a 5-cyclopropyl-2-(4-fluorophenyl)-6-[(2-hydroxy-ethyl)-methanesulfonyl-amino]-benzofuran-3-carboxylic acid methylamide and a therapeutically effective amount of interferon for a time period sufficient to lower the HCV-RNA, wherein the hepatitis C infection is resistant to one of the corresponding monotherapy treatments.

32. A method according to claim 31, wherein said corresponding monotherapy treatment uses 5-cyclopropyl-2-(4-fluoro-phenyl)-6-[(2-hydroxy-ethyl)-methanesulfonyl-amino]-benzofuran-3-carboxylic acid methylamide.

33. A composition for the treatment of hepatitis C infection, comprising an anti-HCV effective amount of 5-cyclopropyl-2-(4-fluoro-phenyl)-6-[(2-hydroxy-ethyl)-methanesulfonyl-amino]-benzofuran-3-carboxylic acid methylamide and an anti-HCV effective amount of interferon.

34. The composition according to claim 33, wherein said interferon is pegylated interferon-alpha-2b.

35. The composition according to claim 33, further comprising at least one supplemental biologically agent selected from the group consisting of ribavirin, protease inhibitors, polymerase inhibitors, small interfering RNA compounds, anti-sense compounds, nucleotide analogs, nucleoside analogs, immunoglobulins, immunomodulators, hepatoprotectants, anti-inflammatory agents, antibiotics, antivirals, and anti-infective compounds.

36. An article of manufacture for the administration of combination therapy to treat hepatitis C infection and associated diseases with therapeutically effective amounts of 5-cyclopropyl-2-(4-fluorophenyl)-6-[(2-hydroxy-ethyl)-methanesulfonyl-amino]-benzofuran-3-carboxylic acid methylamide and interferon, said article of manufacture comprising:
i. a first container containing 5-cyclopropyl-2-(4-fluoro-phenyl)-6-[(2-hydroxy-ethyl)-methanesulfonyl-amino]-benzofuran-3-carboxylic acid methylamide or a pharmaceutically acceptable salt thereof and a first pharmaceutically acceptable carrier medium; and
ii. a second container containing interferon and a second pharmaceutically acceptable carrier medium.

37. The article of manufacture according to claim 36, wherein said first pharmaceutically acceptable carrier medium is suitable for oral administration.
